(19) United States
(12) Patent Application Publication
(10) Pub. No.: US 2023/0110611 A1
(43) Pub. Date: Apr. 13, 2023

(54) USE OF MICROORGANISMS IN REGULATION OF BODYWEIGHT AND CHOLESTEROL LEVEL

(71) Applicant: Microbiota I - Center (Magic) Limited, Shatin, New Territories (CN)

(72) Inventors: Siew Chien NG, Hong Kong (CN); Tao ZUO, Qingzhou (CN); Ka Leung Francis CHAN, Tai Po, New Territories (CN)

(21) Appl. No.: 17/914,250

(22) PCT Filed: Mar. 29, 2021
(86) PCT No.: PCT/CN2021/083553

§ 371 Date: Sep. 23, 2022

Related U.S. Application Data
(60) Provisional application No. 63/001,682, filed on Mar. 30, 2020.

(51) Int. Cl. A61K 35/741; C12Q 1/689; A61K 35/24; A61P 3/06; A61P 3/04

(52) U.S. Cl. CPC A61K 35/741; A61K 35/24; A61P 3/04; A61P 3/06; C12Q 1/689; C12Q 1/6869

(57) ABSTRACT

Certain microorganism species in a composition for use in fecal microbiota transplantation (FMT), including fecal material obtained from an FMT donor, can affect the outcome of FMT treatment such as regulating the bodyweight and cholesterol level in FMT recipients. Thus, methods are provided for identifying subjects as suitable donor to optimize FMT outcome and for pretreating donors and/or recipients for optimized FMT outcome. Also provided are kits and compositions for improving FMT outcome, including for bodyweight and/or cholesterol reduction.

### RELATED APPLICATIONS

This application claims priority to U.S. Provisional Pat. Application No. 63/001,682, filed Mar. 30, 2020, the contents of which are hereby incorporated by reference in the entirety for all purposes.

### BACKGROUND OF THE INVENTION

As living standards continue to improve globally, the number of individuals suffering from diabetes and cardiovascular disease is also rapidly increasing. For example, the World Health Organization (WHO) estimates that by 2030 the number of people living with diabetes will exceed 350 million worldwide. Due to the rising incidence of diabetes and cardiovascular disease, their serious health implications, as well as their profound economic consequences, there exists an urgent need for new and effective means to treat high risk individuals, especially those who are already exhibiting early signs of high likelihood of disease development in the future, such as obesity, higher than normal blood cholestrerol or triglyceride level, inclding higher than normal blood level of low-density lipoprotein cholesterol (LDL-C) and/or lower than normal blood level of high-density lipoprotein cholesterol (HDL-C), so as to reduce or eliminate their risk of later suffering from diabetes and/or cardiovascular disease. The present invention fulfills this and other related needs by providing new methods and compositions that can effectively regulate a patient’s bodyweight and blood cholesterol level.

### BRIEF SUMMARY OF THE INVENTION

The invention relates to novel methods and compositions useful for optimizing fecal microbiota transplantation (FMT) treatment, also known as intestinal microbiota transplantation (IMT), especially for maximizing health benefits conferred to recipients of FMT. In particular, the present inventor discovered that, when certain microorganism species (e.g., bacterial species) are present, especially at an elevated level, in a transplant material for FMT recipient and subsequently in the gastrointestinal (GI, especially intestinal) tract of a recipient after receiving FMT treatment, significant health benefits such as weight loss, higher insulin sensitivity, lower total cholesterol (TC) in blood, lower blood low density lipoprotein cholesterol (LDL-C), higher blood high density lipoprotein cholesterol (HDL-C), and lower blood triglyceride (TG)can be achieved in the FMT recipient. These findings allow the inventors to devise methods and compositions that can improve FMT efficacy in regard to these health benefits. Thus, the present invention provides a novel method for identifying a suitable donor for FMT, who provides fecal material to be used in FMT after proper processing. The method comprising the step of determining the level of one or more bacterial species set forth in Table 2, 3, 4, 5, or 6 in a stool sample obtained from a candidate for FMT donor.

In the first aspect, the present invention provides method for identifying a suitable donor for FMT, comprising the step of determining level of one or more bacterial species set forth in Tables 2-6 in a stool sample obtained from a candidate. In some embodiments, the level of the one or more bacterial species is a percentage relative abundance. In some embodiments, when the level of the one or more bacterial species is found to be greater than their corresponding cut-off value in Tables 2-6, the candidate is identified as a suitable donor for FMT. In some embodiments, the method further comprises a step of obtaining stool material from the suitable candidate for use in FMT. In some embodiments, the method further includes a step of determining total bacterial load in the stool sample.

In some embodiments, the level of one or more bacterial species set forth in Tables 2-6 is determined in a first stool sample obtained from a first candidate and in a second stool sample obtained from a second candidate. In some embodiments, the first candidate has a higher level of each the one or more bacterial species set forth in Tables 2-6 than the second candidate and is deemed to be a more suitable FMT donor than the second candidate. In some embodiments, the first candidate has a higher level of more than half of the one or more bacterial species set forth in Tables 2-6 than the second candidate and is deemed to be a more suitable FMT donor than the second candidate.

In one example, when a donor is screened for his potential as a suitable FMT donor, especially for the purposes of helping a recipient lose weight, the presence of beneficial bacteria such as Bacteroides vulgatus and Alistipes onderdonkii, in his stool sample should reach or surpass a threshold in the relative amount of: e.g., at least about 1% for Bacteroides vulgatus, or with at least about 0.1% of Alistipes onderdonkii. On the other hand, if a candidate donor has in his stool sample inadquate amount of the beneficial bacteria (e.g., less that about 1% for Bacteroides vulgatus and 0.1% for Alistipes onderdonkii), then he should be excluded to serve as a donor, especially when weight reduction is an objective in the proposed FMT process.

In a second aspect, a method is provided for improving FMT efficacy, the method comprising introducing an effective amount of one or more bacterial species set forth in Tables 2-6 into a composition intended for use in transplantation prior to FMT. In some embodiments, after the introducing step the level of each of the one or more bacterial species set forth in Tables 2-6 is greater than a certain percentage of total bacteria in the composition (e.g., greater than the cuf-off value corresponding to each bacterial species). In some embodiments, the method further comprises a step of performing FMT using the composition. In some embodiments, the method further comprises a step of performing FMT using the composition.

In a third aspect, a kit is provided that comprises (1) a first composition comprising donor stool; and (2) a second composition comprising an effective amount of one or more bacterial species set forth in Tables 2-6. In some embodiments, the first composition comprises donor stool that has been dried, frozen, and placed in a capsule for oral ingestion. In some embodiments, the first composition comprises donor stool that has been formulated in the form of a solution, suspension, semi-liquid, or paste for direct delivery via Oesophago-gastro-duodenoscopy (OGD), sigmoidoscopy, or enema. In some embodiments, the kit may comprise printed instructions to guide the user to properly use the kit.

In some embodiments, in the above described methods the level of the one or more bacterial species set forth in Tables 2-6 is determined by quantitative polymerase chain reaction (PCR).

In addition, various methods are provided for therapeutic applications: for example, a method is provided for suppressing blood LDL-C level in a subject, including the step of introducing into the subject’s gastrointestinal tract an effective amount of one or more bacterial species set forth in Tables 3 and 5. In some cases, the introducing step is performed by way of FMT, also known as IMT. Optionally, a step of administering to the individual an effective amount of a broad spectrum anti-bacterial agent such antibiotic is performed before the step of introducing beneficial bacterial species such as FMT/IMT is performed.

Further, a method is provided for suppressing blood total cholesterol in a subject, including a step of introducing into the subject’s gastrointestinal tract an effective amount of one or more bacterial species set forth in Table 4. In some cases, the introducing step is performed by way of FMT such as IMT. Optionally, a step of administering to the individual an effective amount of a broad spectrum anti-bacterial agent such as antibiotic is performed before the step of introducing beneficial bacterial species, such as by FMT or IMT, is performed.

Additionally, a method is provided for suppressing blood triglyceride level in a subject, including a step of introducing into the subject’s gastrointestinal tract an effective amount of one or more bacterial species set forth in Table 6. Optionally, a step of administering to the individual an effective amount of a broad spectrum anti-bacterial agent such as antibiotic is performed before the step of introducing beneficial bacterial species, such as by FMT/IMT, is performed.

Moreover, a method is provided for increasing blood HDL-C level in a subject, including the step of introducing into the subject’s gastrointestinal tract an effective amount of the bacterial species Enterobacter cloacae, for example, by way of FMT/IMT. Optionally, a step of administering to the individual an effective amount of a broad spectrum anti-bacterial agent such as antibiotic is performed before the introducing step, such as by FMT/IMT, is performed.

Lastly, a method is provided for weight reduction in a subject, including the step of introducing into the subject’s gastrointestinal tract an effective amount of one or more bacterial species set forth in Table 2. Optionally, a step of administering to the individual an effective amount of a broad spectrum anti-bacterial agent such antibiotic is performed before the step of introducing beneficial bacterial species set forth in Table 2, such as FMT/IMT, is performed.

For use in any of these methods, a composition comprising an effective amount of any one, two, or more of the beneficial bacterial species set forth in Tables 2-6 is provided, e.g., such a composition may be entirely artificially constituted with a desired quantity of such bacteria; or the composition may be derived from donor stool material that either naturally contains an adequate amount of the bacteria or has been fortified with an added quantity of the bacteria species cultured elsewhere. Donor-derived material typically has been processed prior to use, e.g., dried, frozen, and placed in a capsule for oral ingestion.

### BRIEF DESCRIPTION OF THE DRAWINGS

FIG. 1: The relative abundance of Bacteroides vulgatus and Alistipes onderdonkii in the donors and each recipient before, during, and after IMT. ‘OB’ indicates obese recipient; ‘W’ indicates the n-th week of IMT, where weeks 1-4 represent the 1-month IMT period and weeks 5-16 represent time periods of post-IMT. ‘D’ denotes the n-th day within the indicated week.

FIG. 2: The relative abundance of Alistipes onderdonkii in the duodenum and colon of each IMT recipient during the one-month IMT period. ‘OB’ indicates obese recipient; ‘W’ indicates the n-th week of IMT, where weeks 1-4 represent the 1-month IMT period. ‘D’ denotes the n-th day within the indicated week.

FIG. 3: Schematic diagram of randomized placebo-controlled study of intestinal microbiota transplant (IMT).

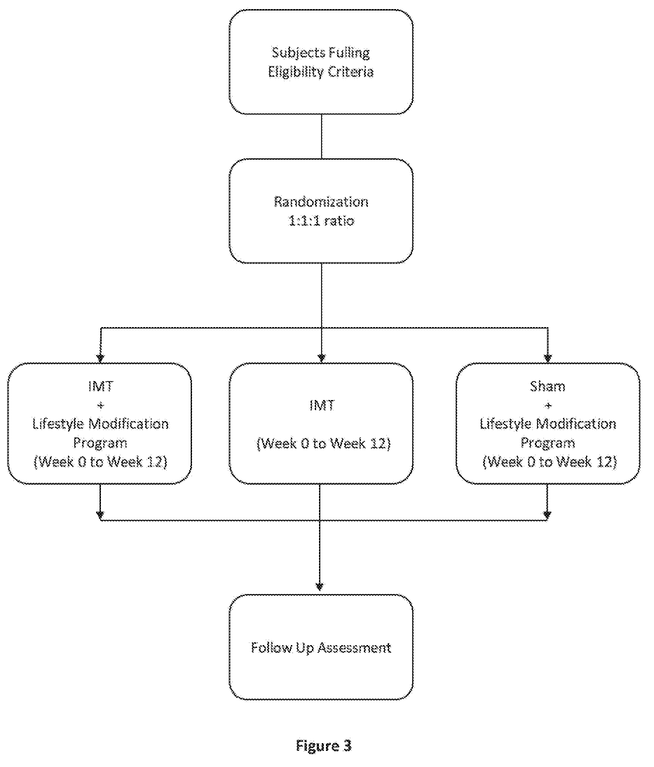

FIG. 4: Mean difference of LDL cholesterol level compared to baseline at different time points after first IMT. Each line represents one randomization arm. At week 12, LDL cholesterol level was measured before receiving the 4^{th} IMT. IMT stopped at week 1.

FIG. 5: Mean difference of total cholesterol level compared to baseline at different time points after first IMT. Each line represents one randomization arm. At week 12, total cholesterol level was measured before receiving the 4^{th} IMT. IMT stopped at week 12.

FIG. 6: The relative abundance of Bacteroides caccae inversely correlate with blood LDL-C and TC (R=-0.39, p<0.01 and R=-0.47, p<0.001, respectively).

FIG. 7: Relative abundance of Bacteroides caccae in the randomized controlled trial. Patients with LDL-C ≤ 1.8 mmol/L throughout the trial are characterized as normal (green); patients with LDL-C > 1.8 mmol/L throughout the trial are characterized as high (blue); patients with LDL-C > 1.8 mmol/L at baseline and LDL-C ≤ 1.8 mmol/L after intervention are characterized as drop (red). Patients with high LDL-C at baseline (W0) have significant lower abundance of Bacteroides caccae compared with patients with normal LDL-C. After intervention, the relative abundance of Bacteroides caccae increased in patients whose LDL-C level returned to normal, while it remained at low level in patients whose LDL-C level remained high.

### DEFINITIONS

The term “fecal microbiota transplantation (FMT)” or “stool transplant” refers to a medical procedure during which fecal matter containing live fecal microorganisms (bacteria, fungi, viruses, and the like) obtained from a healthy individual is transferred into the gastrointestinal tract of a recipient to restore healthy gut microflora that has been disrupted or destroyed by any one of a variety of medical conditions. Typically, the fecal matter from a healthy donor is first processed into an appropriate form for the transplantation, which can be made through direct deposit into the lower gastrointestinal tract such as by colonoscopy, or by nasal intubation, or through oral ingestion of an encapsulated material containing processed (e.g., dried and frozen) fecal matter. One particular form of FMT is intestinal microbiota transplantation or IMT, where the transplanted composition is often delivered by Oesophago-gastro-duodenoscopy (OGD), sigmoidoscopy, or enema. FMT is used for treating a number of medical conditions including obesity, metabolic syndrome, gastrointestinal disorders (such as inflammatory bowel disease (IBD) including ulcerative colitis (UC) and Crohn’s disease (CD)), antibiotic-resistant bacterial infections (such as Clostridium difficile infection (CDI) or conditions caused by multidrug-resistant organisms including carbapenem-resistant Enterobacteriaceae (CRE) or vancomycin-resistant Enterococcus (VRE)), as well as autism, depression, obesity, diabetes, alopecia, acute graft-versus-host disease (aGvHD), and further including certain neurological conditions such as multiple sclerosis and Parkinson’s Disease.

The term “inhibiting” or “inhibition,” as used herein, refers to any detectable negative effect on a target biological process, such as RNA/protein expression of a target gene, the biological activity of a target protein, cellular signal transduction, cell proliferation, presence/level of an organism especially a micro-organism, and the like. Typically, an inhibition is reflected in a decrease of at least 10%, 20%, 30%, 40%, 50%, 60%, 70%, 80%, 90% or greater in the target process (e.g., a subject’s bodyweight, or the blood level of total cholesterol, LCL-C, or triglyceride in a subject), or any one of the downstream parameters mentioned above, when compared to a control. “Inhibition” further includes a 100% reduction, i.e., a complete elimination, prevention, or abolition of a target biological process or signal. The other relative terms such as “suppressing,” “suppression,” “reducing,” and “reduction” are used in a similar fashion in this disclosure to refer to decreases to different levels (e.g., at least 10%, 20%, 30%, 40%, 50%, 60%, 70%, 80%, 90% or greater decrease compared to a control level) up to complete elimination of a target biological process or signal. On the other hand, terms such as “activate,” “activating,” “activation,” “increase,” “increasing,” “promote,” “promoting,” “enhance,” “enhancing,” or “enhancement” are used in this disclosure to encompass positive changes at different levels (e.g., at least about 5%, 10%, 20%, 30%, 40%, 50%, 60%, 70%, 80%, 90%, 100%, 200%, or greater such as 3, 5, 8, 10, 20-fold increase compared to a control level, for example, the control level of one or more of the bacterial species shown in Tables 2-6 or the control level of HDL-C in a subject’s blood) in a target process or signal.

The term “anti-bacterial agent” refers to any substance that is capable of inhibiting, suppressing, or preventing the growth or proliferation of bacterial species. Known agents with anti-bacterial activity include various antibiotics that generally suppress the proliferation of a broad spectrum of bacterial species as well as agents such as antisense oligonucleotides, small inhibitory RNAs, and the like that can inhibit the proliferation of specific bacterial species. The term “anti-bacterial agent” is similarly defined to encompass both agents with broad spectrum activity of killing virtually all species of bacteria and agents that specifically suppress proliferation of target bacteria.

“Percentage relative abundance,” when used in the context of describing the presence of a particular bacterial species (e.g., any one of those shown in any one of Tables 2-6) in relation to all bacterial species present in the same environment, refers to the relative amount of the bacterial species out of the amount of all bacterial species as expressed in a percentage form. For instance, the percentage relative abundance of one particular bacterial species can be determined by comparing the quantity of DNA specific for this species (e.g., determined by quantitative polymerase chain reaction) in one given sample with the quantity of all bacterial DNA (e.g., determined by quantitative polymerase chain reaction (PCR) and sequencing based on the 16s rRNA sequence) in the same sample.

“Absolute abundance,” when used in the context of describing the presence of a particular bacterial species (e.g., any one of those shown in Tables 2-6) in a composition (such as an FMT donor fecal sample), refers to the amount of DNA derived from the bacterial species out of the amount of all DNA in a composition. For instance, the absolute abundance of one bacterium can be determined by comparing the quantity of DNA specific for this bacterial species (e.g., determined by quantitative PCR) in one given sample with the quantity of all fecal DNA in the same sample.

“Total bacterial load” of a composition such as a fecal sample, as used herein, refers to the amount of all bacterial DNA out of the amount of all DNA in the composition (such as a fecal sample). For instance, the absolute abundance of bacteria can be determined by comparing the quantity of bacterial specific DNA (e.g., 16s rRNA determined by quantitative PCR) in one given sample with the quantity of all DNA in the same sample.

The term “effective amount,” as used herein, refers to an amount of a substance that produces a desired effect (e.g., an inhibitory or suppressive effect on a target process, such as change in a subject’s bodyweight, level of total cholesterol, LDL-C, or triglyceride) for which the substance (e.g., one or more beneficial bacterial species in Tables 2-6) is used or administered. The effects include the inhibition or reduction of these levels to any detectable extent. The exact amount will depend on the nature of the substance (the active agent), the manner of use/administration, and the purpose of the application, and will be ascertainable by one skilled in the art using known techniques as well as those described herein. When an “effective amount” of one or more beneficial bacterial species (e.g., those listed in Tables 2-6) are artificially introduced into a composition intended for use in FMT, it is meant that the amount of the pertinent bacteria being introduced is sufficient to confer to the FMT recipient health benefits such as weight loss, improved sensitivity to insulin, reduced blood cholesterol/LDL-C/triglyceride level, and/or increased HDL-C level.

As used herein, the term “about” denotes a range of value that is +/- 10% of a specified value. For instance, “about 10” denotes the value range of 9 to 11 (10 +/- 1).

### DETAILED DESCRIPTION OF THE INVENTION

### I. Introduction

The invention provides a novel approach for assessing the likelihood of effective FMT prior to the procedure being performed as well as for improving the effectiveness of the FMT procedure in conferring certain health benefits (such as bodyweight and cholesterol control) to the recipients. During their studies, the present inventors discovered that the presence and relative abundance of certain bacterial species both in a recipient’s gastrointestinal tract and in a composition intended for use in FMT (such as donor’s stool) directly correlate with the outcome of FMT. For example, the presence of bacterial species shown in Tables 2-6, especially at an elevated level, are found to confer health benefits to the FMT recipients, such as inducing weight loss, reducing blood cholesterol level, LDL-C level, and/or triglyceride level, as well as increasing blood HLDL-C level in the receipients. Analysis of the level of these pertinent species of bacteria in a proposed FMT donor stool can determine whether the individual is an appropriate FMT donor or which individual among two or more candidate donors would serve as the best donor for FMT in order to optimize the therapeutic outcome, especially the above-named health benefits.

### II. FMT Donor/Recipient Selection and Preparation

Patients suffering from Clostridium difficile infection (CDI), especially recurring CDI, are often considered as recipients for FMT treatment. Aside from CDI, other diseases and conditions, including those of digestive system or nervous system such as colitis, irritable bowel syndrome (IBS), Crohn’s disease, acute graft-versus-host disease (aGvHD), infections caused by multidrug-resistant bacteria such as CRE or VRE, multiple sclerosis, Parkinson’s Disease, diabetes mellitus, and obesity are also suitable for FMT treatment.

Fecal matter used in FMT is obtained from a healthy donor and then processed into appropriate forms for the intended means of delivery in the upcoming FMT procedure. Up until very recently, the general criterion for an FMT donor has been simply that the donor is a healthy individual without any known diseases or disorders especially in the digestive tract, although some preference is often given to the members of the same household as the recipient.

The present inventors have discovered in their studies that elevated presence of one or more “beneficial” bacterial species, such as those shown in Tables 2-6, in a recipient’s gastrointestinal tract or in a donor stool (which is used in the transplantation after being processed) can confer significant health benefits following FMT treatment in a patient, such as bodyweight loss, improved insulin sensitivity, reduced blood/serum/plasma cholesterol (especially LDL-C) or triglyceride level, as well as increased blood/serum/plasma cholesterol HDL-C level in the recipient.

This revelation enables the initial screening of individuals as appropriate FMT donors as well as the initial screening of patients as likely candidates for successful and beneficial FMT treatment, especially in the case of treatment for obesity or metabolic syndrome including insulin insensitivity and/or type II diabetes: if a candidate donor’s stool contains a minimal or elevated level of any one or multiple bacterial species shown in Tables 2-6 (e.g., each is greater than the cut-off value of about 0.004%, 0.005%, 0.006%, 0.007%, 0.0075%, 0.0078%, 0.008%, 0.01%, 0.012%, 0.1%, 0.115%, 0.147%, 0.2%, 0.25%, 0.255%, 0.4%, 0.6%, 0.8%, 1%, 1.2%, 1.5%, 1.8%, 2%, 2.021%, 2.5%, 2.8%, 2.864%, or 3% of total bacteria), the candidate is deemed as suitable as an FMT donor, and his stool can immediately retrieved for processing and later used in FMT; on the other hand, if a candidate’s stool sample shows no or only low level of these beneficial bacterial species (e.g., each is no greater than the cut-off value of about 0.004%, 0.005%, 0.006%, 0.007%, 0.0075%, 0.0078%, 0.008%, 0.01%, 0.012%, 0.1%, 0.115%, 0.147%, 0.2%, 0.25%, 0.255%, 0.4%, 0.6%, 0.8%, 1%, 1.2%, 1.5%, 1.8%, 2%, 2.021%, 2.5%, 2.8%, 2.864%, or 3% of total bacteria), then the candidate is deemed not an immediately appropriate FMT donor and his fecal material should not be taken for use in FMT without necessary modification before potential use. One possible means of improving donor fecal material prior to processing for use in FMT is to artificially introduce one or more of the beneficial bacterial species (e.g., those in Tables 2-6), which may have been artificially cultured and then concentrated, isolated, enriched, or purified, so as to increase the presence of such bacterial species in the fecal material for use in FMT (e.g., each species is greater than the cut-off value of about 0.004%, 0.005%, 0.006%, 0.007%, 0.0075%, 0.0078%, 0.008%, 0.01%, 0.012%, 0.1%, 0.115%, 0.147%, 0.2%, 0.25%, 0.255%, 0.4%, 0.6%, 0.8%, 1%, 1.2%, 1.5%, 1.8%, 2%, 2.021%, 2.5%, 2.8%, 2.864%, or 3% of total bacteria).

Ideally, a desirable FMT composition prepared from a donor fecal material and intended for use in FMT for treating obesity or metabolic syndrome or type II diabetes or elevated/unhealthy high level of cholesterol, LDL-C, and triglyceride has both a high level of one or more beneficial bacterial species (e.g., those shown in Tables 2-6). Thus, while modifications can be made to a composition intended for use in FMT to address the insufficient level of beneficial bacteria, one possible modification is to increase the level of one or more beneficial bacterial species (e.g., those shown in Tables 2-6), for example, by supplementing the transfer material intended for use in FMT or by directly introducing into the recipient’s GI tract an adequate amount of such beneficial bacterial species, optionally while at the same time to suppress the level of one or more detrimental bacterial species (e.g., those shown in Table 1b) in order to maximize the potential health benefits a recipient may derived from the FMT procedure.

Various methods have been reported in the literature for determining the levels of all bacterial species in a sample, for example, amplification (e.g., by PCR) and sequencing of bacterial polynucleotide sequence taking advantage of the sequence similarity in the commonly shared 16S rDNA bacterial sequences. On the other hand, the level of any given bacterial species may be determined by amplification and sequencing of its unique genomic sequence. A percentage abundance is often used as a parameter to indicate the relative level of a bacterial species in a given environment.

### III. Treatment Methods Using Beneficial Microorganisms

The discovery by the present inventors reveals the direct correlation between (1) certain “beneficial” bacterial species in an individual’s stool or GI tract or in the transfer material derived from donor stool for use in FMT and (2) presence/absence of significant health benefits conferred by way of FMT treatment to an FMT recipient, such as body weight loss, improved insulin sensitivity, lowered blood cholesterol/LDL-C/triglyceride level, and increased HDL-C level. This discovery not only allows one to devise an initial screening process to identify appropriate donors and recipients to secure therapeutic efficacy and/or health benefits from the FMT procedure, it also enables different methods for enhancing or optimizing the potential health benefits conferred by the FMT procedure through modulating (increasing or decreasing) the level of one or more of the beneficial bacterial species shown in Tables 2-6 here in a donor stool material and in a recipient prior to the FMT treatment.

As discussed in the above section, when a proposed FMT donor whose stool is tested and found to contain an insufficient level of one or more of the beneficial bacterial species such as those shown in Tables 2-6 (e.g., each is less than the corresponding cut-off value of about 0.004%, 0.005%, 0.006%, 0.007%, 0.0075%, 0.0078%, 0.008%, 0.01%, 0.012%, 0.1%, 0.115%, 0.147%, 0.2%, 0.25%, 0.255%, 0.4%, 0.6%, 0.8%, 1%, 1.2%, 1.5%, 1.8%, 2%, 2.021%, 2.5%, 2.8%, 2.864%, or 3% of total bacteria in the stool sample), the proposed donor is deemed as an unsuitable donor for FMT intended to confer health benefits such as reduced body weight, sensitized response to insulin, reduced blood cholesterol/LDL-C/triglyceride level, and increased HDL-C level, he may be disqualified as a donor in favor of anther individual whose stool sample exhibits a more favorable bacterial profile, and his fecal material should not be immediately used for FMT due to the lack of prospect of conferring such beneficial health effects unless the stool material is adequately modified. In these cases of expected lack of health benefits from FMT treatment can be readily improved in view of the inventors’ discovery, for example, one or more of the bacterial species shown in Tables 2-6 may be introduced from an exogenous source into a donor fecal material so that the level of the bacterial species in the fecal material is increased (e.g., to reach at least the corresponding cut-off value of about 0.004%, 0.005%, 0.006%, 0.007%, 0.0075%, 0.0078%, 0.008%, 0.01%, 0.012%, 0.1%, 0.115%, 0.147%, 0.2%, 0.25%, 0.255%, 0.4%, 0.6%, 0.8%, 1%, 1.2%, 1.5%, 1.8%, 2%, 2.021%, 2.5%, 2.8%, 2.864%, or 3% of total bacteria in the fecal material) before it is processed for use in FMT for the treatment of obesity, metabolic syndrome, type II diabetes, or unhealthy high levels of cholesterol/LDL-C/triglyceride. Pretreatment schemes with similarly intended goals can be employed to prepare patients who are soon to receive FMT treatment in order to maximize their potential to receive health benefits such as those stated above and herein.

Immediately upon completion of the FMT procedure, the recipient may be further monitored by continuous testing of the level of beneficial bacterial species in the stool samples on a daily basis for up to 5 days post-FMT while the clinical symptoms of the condition being treated as well as the intended health benefits (e.g., bodyweight, blood cholesterol, LDL-C, triglyceride, or HDL-C levels) are also being monitored in order to assess FMT outcome and the corresponding levels of relevant bacteria in the recipient’s GI tract: in the case of treating obesity/patient weight manipulation, the level of bacterial species set forth in Tables 2-6 may be monitored in connection with observation of health benefits achieved such as weight loss, insulin sensitivity improvement, blood cholesterol/LDL-C/triglyceride reduction, and increased blood HDL-C level.

### IV. Kits and Compositions for Improved FMT

The present invention also provides novel kits and compositions that can be used for improving therapeutic efficacy and health benefits delivered by various therapeutic and/or prophylactic treatment schemes involving FMT. For example, in a kit for treating a patient in need of FMT (e.g., for obesity/body weight control, reduction of blood cholesterol/LDL-C/triglyceride level, increase of blood HDL-C level), a first composition intended for transplantation into a patient or FMT recipient and a second composition for either increasing the level of one or more of the beneficial bacterial species (such as those shown in Tables 2-6)—this composition may be in tended to be added to the first composition or it may be intended to be administrated to the recipient, e.g., directly deposited in the GI tract, such as delivery in the intestinal tract by oesophago-gastro-duodenoscopy (OGD), sigmoidoscopy, or enema. The first composition comprises a fecal material from a donor, which has been processed, formulated, and packaged to be in an appropriate form in accordance with the delivery means in the FMT procedure, which may be by direct deposit in the recipient’s lower gastrointestinal track (e.g., wet or semi-wet form) or by oral ingestion (e.g., frozen dried encapsulated). The second composition in some cases may comprises an adequate or effective amount of one or more of the beneficial bacterial species (such as those shown in Tables 2-6), such that it can be added to the first composition prior to FMT for the purpose of optimizing the prospect of achieving therapeutic efficacy and/or conferring health benefits to the recipient. The composition is formulated for the intended delivery method, for example, by oral ingestion or by local deposit (e.g., suppositories). The first and second compositions are often kept separately in two different containers in the kit. In some cases, aside from the composition for increasing the beneficial bacterial species, there is an additional composition comprising a broad spectrum anti-bacteria agent, and they are provided in separate containers as the second and third components of the kit. Typically, the kit will further include printed material providing detailed instructions for users of the kit, such as providing information of the schedule and dosing arrangement for administering the first and second (and optionally third) compositions to a recipient.

In another aspect of this invention, alternative compositions useful in FMT with improved efficacy may be devised to contain at least these two components: (1) a donor stool material containing live fecal microorganisms, and (2) an anti-bacterial agent that generally suppresses the growth or proliferation of virtually all bacterial species. Such broad spectrum anti-bacteria agent may be used to suppress all bacterial species in the GI tract of a pre-FMT patient in preparation for the FMT treatment in order to optimize the beneficial health effects of one or more of the bacterial species set forth in Tables 2-6.

### EXAMPLES

The following examples are provided by way of illustration only and not by way of limitation. Those of skill in the art will readily recognize a variety of non-critical parameters that could be changed or modified to yield essentially the same or similar results.

### Introduction

The purpose of this invention is to determine how human gut fungome and virome is associated with high density lipoprotein cholesterol (HDL-C) and may impact diseases related with low HDL-C. HDL-C is a favourable cholesterol and higher level has been associated with lower risk of cardiovascular diseases. The practical use of the invention includes improving human health and opposing disease risks associated with low HDL-C, such as obesity, cardiovascular diseases, hypertension and diabetes, by modulating human gut fungome and virome. These measures may include fecal microbiota transplantation (FMT) with optimized protocols, synthetic fungal and/or viral species supplementation, countermeasures for clearing microorganisms to decrease or to treat low HDL-C related diseases. This will promote development microbial product and add up a set of criteria for establishment of stool bank and its derived products in diagnostics and therapeutics.

### Example 1. Open-Label Clinical Trial of IMT in Patients with Obesity

### Methods

### Study Design

An open-label clinical trial of intestinal microbiota transplant (NCT03789461) in obese subjects was conducted. Subjects aged 18-75, has a body mass index (BMI) ≥28 kg/m² and < 45 kg/m² and with informed consent obtained were recruited. During study, subjects received intensive IMT for a total of 20 days. Every week during treatment period, subject received 5 days of IMT (5 days on and 2 days off). During the same period, subjects also received dietary and lifestyle advice. After IMT, the subjects were followed up at weeks 6, 8, 12, 16. Subjects’ fecal samples were collected. In addition, duodenum and colon biopsies were taken from subjects during IMT.

### Intestinal Microbiota Transplantation (IMT)

Prior to receiving IMT, subjects received 5 days of antibiotics consisting of Vancomycin 500 mg 3 times daily, Metronidazole 500 mg 3 times daily and Amoxicilin 500 mg 3 times daily to enhance the engraftment of the microbiota from IMT. Then subjects received 20 days of IMT. 100-200 ml of IMT solution were infused to patients via standard procedures including Oesophago-gastro-duodenoscopy (OGD), sigmoidoscopy, or enema in either in-patient or out-patient settings.
1. Via OGD: 100-200 ml of IMT solution were infused over 2-3 minutes into the distal duodenum or jejunum via OGD. After infusion, subjects were monitored for 1 hour before discharge.
2. Via Sigmoidoscopy: 100-200 ml of IMT solution were infused over 2-3 minutes into the distal colon via sigmoidoscopy. After infusion, subjects were monitored for 1 hour before discharged.
3. Via Enema: 100-150 ml of IMT solution were self-administered or with help from research team via enema. Subjects were instructed to retain the enema for 20-30 minutes. 4 mg loperamide were given before each enema to enhance the retention of IMT solution.

### Stool Donors of Stool Bank of the Chinese University of Hong Kong

Donors (BMI < 23 kg/m²) were volunteers from general population including spouses or partners, first-degree relatives, other relatives, friends and others who are known or unknown to the potential subjects. Potential donors who met eligibility criteria were invited for screening laboratory test. A series of laboratory tests for infectious diseases and interviews were done. Stool from the eligible donors were used in this study. All subjects received stools from a single donor in this study.

### Dietary and Lifestyle Advice

Subjects were contacted by a research dietitian and trained research personnel in every treatment visit. Subjects were guided in terms of their dietary habits, physical activity patterns, and other lifestyle habits. Diet history were collected in the initial assessment and follow ups. Subjects completed a 24-hour diet recall for the first four weeks; thereafter, a 3 day dietary record every other week up to 26 weeks. These were done face-to-face or by phone. Phone follow-ups were conducted on the alternate weeks up to 26 weeks and every 26 weeks for the remaining study period. Subjects also received dietary supplements such as Metamucil (Psyllium husk).

### Demographics and Medical History

Demographics and medical history such as sex, age, smoking and alcohol status, disease onset, co-morbid illness, drug history, clinical test results were obtained by reviewing of subject medical notes and interview with subjects by doctors and research staff.

### Obesity Measurement

Body weight, height, BMI, waist and hip circumference, waist to hip ratio, blood pressure, and heart rate were measured.

### Routine Blood Test

Blood samples were collected for complete blood count (CBC), renal function test (RFT), liver function test (LFT), C-reactive protein (CRP) and magnesium. Fasting serum concentrations of glucose, insulin (only at baseline and week 4), total cholesterol, low-density-lipoprotein (LDL) cholesterol, high-density-lipoprotein (HDL) cholesterol, triglycerides and haemoglobin Alc (HbAlC) were measured. For baseline, blood samples were collected within 7 days before first IMT.

### Clinical Laboratory Test

Fasting blood samples were collected before IMT and at week 4, 12, 16, 20, and 24, which were tested complete blood count (CBC), renal function test (RFT), liver function test (LFT), C-reactive protein (CRP), magnesium, glucose, insulin (only before IMT and week 24), total cholesterol, low-density-lipoprotein (LDL) cholesterol, high-density-lipoprotein (HDL) cholesterol, triglycerides and haemoglobin Alc (HbAlC).

### Statistical Methods

The statistical analyses were done using SPSS. Repeated measure ANOVA was used to determine the difference between baseline, week 12, week 16, week 20, and week 24 follow-up within the three treatment arms. The difference between baseline and each time points was calculated. Post-hoc analysis was done using Bonferroni test. p-value of <0.05 was considered significant.

### DNA Extraction

Fecal and mucosal DNA was extracted by using Maxwell® RSC PureFood GMO and Authentication Kit (Promega). Approximately 100 mg stool sample or mucosal biopsy was suspended in 800 µL TE buffer (pH 7.5), supplemented with 1.6 µl 2-mercaptoethanol and 500 U lyticase (Sigma) and incubated at 37° C. for 60 min. The sample was then centrifuged at 13,000×g for 2 min and the supernatant was discarded. After this pretreatment, DNA was subsequently extracted from the pellet using a Maxwell® RSC PureFood GMO and Authentication Kit (Promega) following manufacturer’s instructions. Briefly, 1 ml of CTAB buffer was added to the pellet and vortexed for 30 s, then the solution heated at 95° C. for 5 min. After that, samples were vortexed thoroughly with beads (Biospec, 0.5 mm for fungi and 0.1 mm for bacteria, 1:1) at maximum speed for 15 min. Following this, 40 µl proteinase K and 20 µl RNase A were added and the mixture incubated at 70° C. for 10 min. The supernatant was then obtained by centrifuging at 13,000×g for 5 min and placed in a Maxwell® RSC instrument for DNA extraction. The extracted fecal DNA was used for ultra-deep metagenomics sequencing via Ilumina Novoseq 6000 (Novogen, Beijing, China).

### Quality Filtering Metagenome Sequence Data

Raw sequence reads were filtered and quality-trimmed using Trimmomatic v0.36¹ as follows: 1) Trimming low quality base (quality score < 20); 2) Removing reads shorter than 50 bp; 3) removing sequences less than 50 bp long; 4) Tracing and cutting off sequencing adapters. Contaminating human reads were filtering using Kneaddata (Reference database: GRCh38 p12) with default parameters.

### Profiling the Bacterial Microbiome from the Metagenomic Dataset

Profiling of bacterial microbiome (bacteriome) was performed via MetaPhlAn2 by mapping reads to clade-specific markers² and annotation of species pangenomes through Bowtie2 was downloaded and used as reference to align clean sequence reads using Bowtie2³. Correlation between patient post-IMT bodyweight decrease and the bacterial microbiome composition was analyzed by Spearsman’s correlation estimation.

### 16s RRNA Sequencing and Quality Control

The extracted mucosal DNA samples were sequenced on the Illumina Hiseq 2500 platform (V3-V4 region, 2 × 250 bp). Quality control and data analysis were implemented in mothur (v 1.38.0) as previously describe. Any sequences with ambiguous bases and anything longer than 275 bp were removed, and aligned against the non-redundant Greengenes database (v 13.8) using the NAST algorithm. Any sequences that failed to align with the V3-4 region were discarded. The remaining sequences were trimmed to the same alignment coordinates over which they fully overlapped, followed by removal of homopolymers and detection for the presence of chimeras by UChime.

### 16s RRNA Sequencing Data Analysis

The resulting sequences were classified against the Greengenes database and annotated with deepest level taxa represented by pseudo-bootstrap confidence scores of at least 80% averaged over 1,000 iterations of the naive Bayesian classifier. Any sequences that were classified as either being originated from archaea, eukarya, chloroplasts, mitochondria, or unknown kingdoms, were removed. The annotated sequences were assigned to phylotypes according to their consensus taxonomy with which at least 80% of the sequences agreed.

### Results

### Bodyweight Reduction

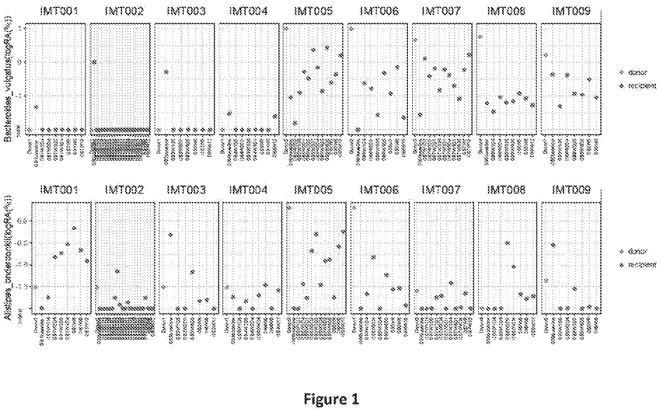

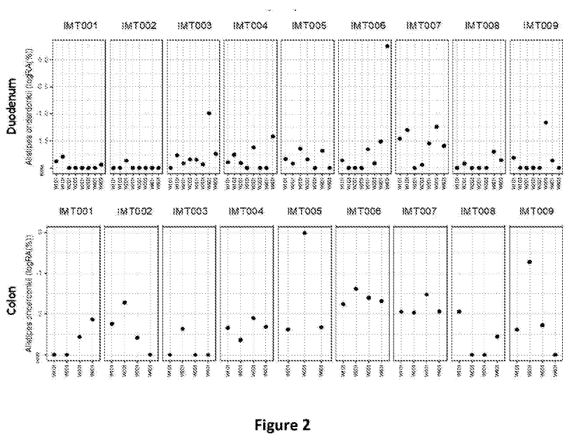

Nine subjects were recruited. All nine subjects showed bodyweight loss after IMT (Table 1). During post-IMT follow-up, subjects IMT5, IMT6, and IMT7 showed the most significant body weight loss among all IMT recipients. IMT1 showed modest and sustainable bodyweight loss after IMT. Correlation analysis between post-IMT body weight loss and the fecal bacteria profile identified the species Bacteroides vulgatus and Alistipes onderdonkii as the prominent species associated with bodyweight reduction (FIG. 1). Their respective correlation coefficients with bodyweight loss were -25.89 and -96.34. In accordance, Bacteroides vulgatus showed higher presence in post-IMT stools of IMT5, IMT6, and IMT7 (who had the most marked body weight loss) relative to other recipients (FIG. 1). Alistipes onderdonkii showed higher presence in post-IMT stools of IMT1 and IMT5 relative to other recipients (FIG. 1). In addition, Alistipes onderdonkii showed higher presence at the mucosa (both the duodenum and colon) of IMT6 and IMT7 relative to other recipients (FIG. 2). These data indicate that Bacteroides vulgatus (NCBI taxon ID 821) and Alistipes onderdonkii (NCBI taxon ID 328813) play an important role in reducing obesity.

TABLE 1

| Bodyweight Change of Obese Subjects after Receiving Intestinal Microbiota Transplantation (IMT) | | | | | | | | | |
|---|---|---|---|---|---|---|---|---|---|
| IMT Recipient | IMT001 | IMT002 | IMT003 | IMT004 | IMT005 | IMT006 | IMT007 | IMT008 | IMT009 |
| Sex | M | M | F | M | F | F | F | F | M |
| Stool Donor | D8 | D8 | D8 | D8 | D16 | D16 | D15 | D18 | D19 |
| Initial weight (kg) | 113.4 | 127.1 | 95.5 | 98.5 | 111.6 | 108.9 | 98.3 | 84.1 | 82.8 |
| Bodyweight change (kg) | | | | | | | | | |
| Week 4 | -4.7 | -1.9 | -0.5 | -2.9 | -2.7 | -2.5 | -2.8 | -3.9 | -1.1 |
| Week 6 | -2.7 | -1.6 | -2.3 | -1.1 | -3.7 | -1.6 | Defaulted | -3.3 | 0.1 |
| Week 8 | -3.2 | 1.3 | -1.8 | -0.9 | -4.2 | -4.2 | -5.4 | -1.5 | -1.2 |
| Week 12 | -3 | 0.4 | -0.9 | -0.9 | -3.3 | -3.2 | -8.1 | -2.7 | -0.9 |
| Week 16 | -2.8 | 0.4 | -0.7 | -1.4 | -5.2 | -4.1 | -3.3 | -1 | -0.9 |

TABLE 2

Bacterial species for reduction of bodyweight

| Bacterial Species | NCBI Taxon ID | Cuf-off (relative abundance) |
|---|---|---|
| Bacteroides vulgatus | 821 | 1% |
| Alistipes onderdonkii | 328813 | 0.1% |

### Reduction in Low-Density-Lipoprotein Cholesterol (LDL-C) Level

Optimal total cholesterol level is about 150 mg/dL (3.8 mmol/L), corresponding to an LDL-C level of about 100 mg/dL (2.6 mmol/L)⁴. Two subjects (IMT003 and IMT007), who had an elevated level of LDL-C of 3.6 mmol/L at baseline, showed marked reduction of LDL-C after receiving 20 days of IMT. IMT003 is a 42-year-old female. Compared with the baseline, LDL-C level of IMT003 was reduced by 8.33% at both week 3 and week 6, and reduced by 11.11% at week 16. IMT007 is a 34-year-old female, and her LDL-C level showed 5.56% and 19.44% reduction at week 3 and week 6, respectively, compared to the baseline.

### Example 2. Cross-Sectional Study of Healthy Chinese

### Methods

### Cohort Descritpion and Study Subjects

A total of 942 healthy Chinese were recruited. The study was approved by The Joint Chinese University of Hong Kong, New Territories, East Cluster Clinical Research Ethics Committee (The Joint CUHK-NTEC CREC, CREC Ref. No: 2016.407), and by the Institutional Review Board (IRB) and Research Ethics Committee of the first affiliated hospital of Kunming Medical School (Ref. No: 2017.L.14). All subjects consented to donate fecal samples as well as blood samples and to the questionnaire investigation, where written informed consents were obtained. Fecal samples from the study subjects were stored at -80° C. for downstream microbial analyses.

### Fecal DNA Extraction and DNA Sequencing

Fecal DNA was extracted by using Maxwell® RSC PureFood GMO and Authentication Kit (Promega). Approximately 100 mg from each stool sample was prewashed with 1 ml ddH₂O and pelleted by centrifugation at 13,000 ×g for 1 min. The pellet was resuspended in 800 µL TE buffer (pH 7.5), supplemented with 1.6 µl 2-mercaptoethanol and 500 U lyticase (Sigma), and incubated at 37° C. for 60 min, which increase the lysis efficacy of fungal cell. The sample was then centrifuged at 13,000×g for 2 min and the supernatant was discarded. After this pretreatment, DNA was subsequently extracted from the pellet using a Maxwell® RSC PureFood GMO and Authentication Kit (Promega) following manufacturer’s instructions. Briefly, 1 ml of CTAB buffer was added to the pellet and vortexed for 30 s, then the solution heated at 95° C. for 5 min. After that, samples were vortexed thoroughly with beads (Biospec, 0.5 mm for fungi and 0.1 mm for bacteria, 1:1) at maximum speed for 15 min. Following this, 40 µl proteinase K and 20 µl RNase A were added and the mixture Incubated at 70° C. for 10 min. The supernatant was then obtained by centrifuging at 13,000×g for 5 min and placed in a Maxwell® RSC instrument for DNA extraction. The extracted fecal DNA was used for ultra-deep metagenomics sequencing via Ilumina Novoseq 6000 (Novogen, Beijing, China). An average of 52 ± 6.3 million reads (12 G clean data) per sample were obtained.

### Quality Trimming of Raw Sequences

Raw sequence reads were filtered and quality-trimmed using Trimmomatic v0.36¹ as follows: 1) Trimming low quality base (quality score < 20); 2) Removing reads shorter than 50 bp; 3) removing sequences less than 50 bp long; 3) Tracing and cutting off sequencing adapters. Contaminating human reads were filtering using Kneaddata (Reference database: GRCh38 p12) with default parameters.

### Profiling of the Microbiome

Profiling of bacterial microbiome (bacteriome) was performed via MetaPhlAn2 by mapping reads to clade-specific markers² and annotation of species pangenomes through Bowtie2 ³. The resulting bacterial species abundance table was used to correlate to the blood parameters. Pearson correlation and spearman correlations and P values were calculated using cor and cor.test functions in R and visualized using the ggplot2.

### Results

### Gut Bacteria Correlate with Blood Parameters in Humans

The human gut bacteria composition was studied and the abundances of bacterial species were correlated with the blood parameters across all healthy subjects. Among all probed blood parameters, lipid metabolism related parameters, such as triglyceride, total cholesterol, high density lipoprotein-cholesterol (HDL), low density lipoprotein-cholesterol (LDL-C), and glucose, were of special interest as their levels are closely associated with the risk of metabolic disease.

The species Adlercreutzia equolifaciens, Collinsella aerofaciens, Weissella cibaria, Weissella confusa, Lactococcus garvieae, Lactococcus lactis, Butyrivibrio crossotus, Roseburia hominis, and Ruminococcus callidus were discovered to inversely correlate with lipid metabolism parameters (total cholesterol, triglyceride, LDL-C), indicating these species are putative salutary microbes protecting host against metabolic diseases and cardiovascular disease. In addition, the species Enterobacter cloacae showed both from inverse correlation with total cholesterol, LDL-C and triglyceride, and positive correlation with HDL-C (Table 3), indicating that this species is also a salutary microbe protecting against metabolic diseases and cardiovascular disease.

TABLE 3

Bacterial species for reduction of low-density lipoprotein cholesterol (LDL-C)

| Bacterial Species | NCBI Taxon ID | Cut-off (relative abundance) |
|---|---|---|
| Adlercreutzia equolifaciens | 446660 | 0.147% |
| Collinsella aerofaciens | 74426 | 2.864% |
| Weissella cibaria | 137591 | 0.01% |
| Weissella confusa | 1583 | 0.01% |
| Lactococcus garvieae | 1363 | 0.01% |
| Lactococcus lactis | 1358 | 0.01% |
| butyrivibrio crossotuse | 45851 | 0.01% |
| Roseburia hominis | 301301 | 0.078% |
| Ruminococcus callidus | 40519 | 0.01% |
| Enterobacter cloacae | 550 | 0.01% |

### Example 3: Randomized Placebo-Controlled Trial of Intestinal Microbiota Transplantation in Subjects with Obesity and Diabetes Mellitus

### Methods

### Study Design

A randomized placebo-controlled study of intestinal microbiota transplant (IMT) was conducted in obese subjects with type 2 diabetes mellitus (NCT03127696). Consented subjects who fulfil eligibility criteria in Prince of Wales Hospital were recruited. Inclusion criteria include age between 18 to 70, BMI ≥ 28 kg/m² and < 45 kg/m²; and having a diagnosis of Type 2 diabetes mellitus for ≥3 months. Exclusion criteria include current pregnancy, use of any weight loss medications in the preceding 1 year, known history or concomitant significant gastrointestinal disorders (including Inflammatory Bowel Disease, current colorectal cancer, current GI infection), known history or concomitant significant food allergies, immunosuppressed subjects, known history of severe organ failure (including decompensated cirrhosis), inflammatory bowel disease, kidney failure, epilepsy, acquired immunodeficiency syndrome, current active sepsis, active malignant disease in recent 2 years, known contraindications to oesophago-gastro-duodenoscopy (OGD), use of probiotic or antibiotics in recent 3 months, on Sodium-glucose co-transporter-2 inhibitors or Glucagon-like peptide-1 receptor agonists at randomization, or on Proton-pump inhibitor at randomization. Subjects were randomized to 3 arms in 1:1:1 ratio (Arm 1: IMT and lifestyle modification programme, Arm 2: IMT alone, Arm 3: Sham and lifestyle modification programme) (FIG. 2).

### Prohibited Medications

No antibiotics, probiotic or prebiotic preparations, Sodium-glucose co-transporter-2 (SGLT2) inhibitors, Glucagon-like peptide-1 (GLP-1) receptor agonists or Proton-pump inhibitor (PPI) were permitted during the study.

### Intestinal Microbiota Transplant (IMT)/Sham Infusion

Subjects received IMT / Sham infusion 4 times at week 0, 4, 8, and 12 and were followed up until week 24. Each time, 100-200 ml of IMT / sham solution were infused over 2-3 minutes into the distal duodenum or jejunum via OGD. IMT and sham solution were prepared as follow.

IMT: Frozen stool from donors of stool bank were used. For each IMT, IMT solution was infused using stool from single donor or mixing of stool from multiple donors. IMT solution was prepared by diluting feces with sterile saline (0.9%). This solution was blended and strained with filter. The resulting supernatant was then stored as frozen IMT solution for later use.

Sham: Sterile saline (0.9%) were used as sham.

### Stool Donors of Stool Bank of the Chinese University of Hong Kong

Donors (BMI < 23 kg/m²) were volunteers from general population including spouses or partners, first-degree relatives, other relatives, friends and others who are known or unknown to the potential subjects. Potential donors who met eligibility criteria will be invited for screening laboratory test. A series of laboratory tests for infectious diseases and interviews were done. Stool from the eligible donors were used in this study. Subjects may receive stools from single or multiple donors whose identity may not be made available to the subjects.

### Results

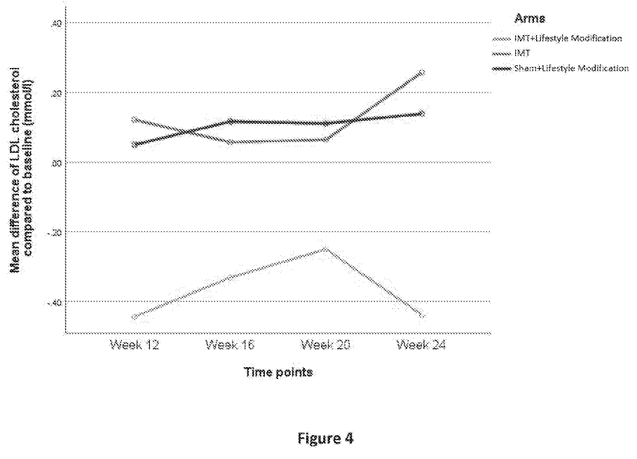

61 subjects were recruited and randomly assigned to IMT and lifestyle modification programme (LSM, n=21), IMT alone (n=20) and sham and lifestyle modification programme (LSM, n=20). Four subjects withdrew from the study. Results showed a significant difference in the LDL cholesterol level three months after treatment between the three treatment arms (p=0.001, repeated measure ANOVA). Post-hoc analysis showed that LDL cholesterol level of the IMT with lifestyle modification programme arm was significantly lower than that of IMT alone (p=0.003) and sham with lifestyle modification programme (p=0.003). The reduction of LDL cholesterol level in IMT with lifestyle modification programme arm was observed at week 12 (after 3 IMT), and maintained at a level below baseline even after stopping IMT. Such effect was observed until last follow up at week 24 (FIG. 4).

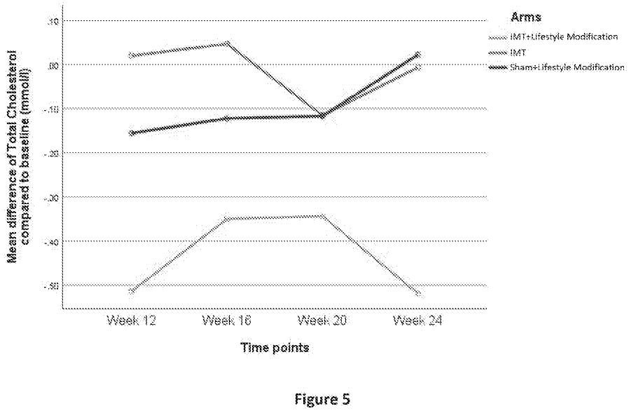

There was also a significant difference in the total cholesterol level between three treatment arms (p=0.021, repeated measure ANOVA). Post-hoc analysis showed that total cholesterol was significantly lower in the arm receiving IMT with lifestyle modification programme, when compared to the arm with IMT alone (p=0.029). Similar to the LDL cholesterol level, reduction of total cholesterol level in IMT with lifestyle modification programme arm was observed since week 12. Although total cholesterol in IMT with lifestyle modification programme arm increased at week 16 and week 20, the overall trend was still below baseline (FIG. 5). There was no significant difference in HDL cholesterol and triglycerides between randomization arms.

In IMT with lifestyle modification arm, LDL cholesterol level reduced 14.4%, 8.7%, 5.9% and 10.1% at week 12, week 16, week 20 and week 24 respectively. In the IMT alone arm (without lifestyle modification), 3 subjects (FDM017, FDM026 and FDM060) receiving showed reduction of LDL-C. Compared to baseline, FDM017, 59-year-old male, showed 28% reduction of LDL cholesterol level at week 24. FDM026, 36-year-old male, showed 18% reduction of LDL-C at week 24. FDM060, 45-year-old female, showed 13% reduction of LDL cholesterol at week 24.

It was discovered that the species Megasphaera micronuciformis, Klebsiella pneumoniae inversely correlate with LDL, and Lactobacillus mucosae, Lactobacillus rhamnosus inversely correlate with TC, while Bacteroides caccae (a fiber-degrading symbiont common in microbiomes of Western individuals)⁵ inversely correlates with both TC and LDL, indicating these species are putative salutary microbes protecting host against metabolic diseases and cardiovascular disease (Tables 4 and 5).

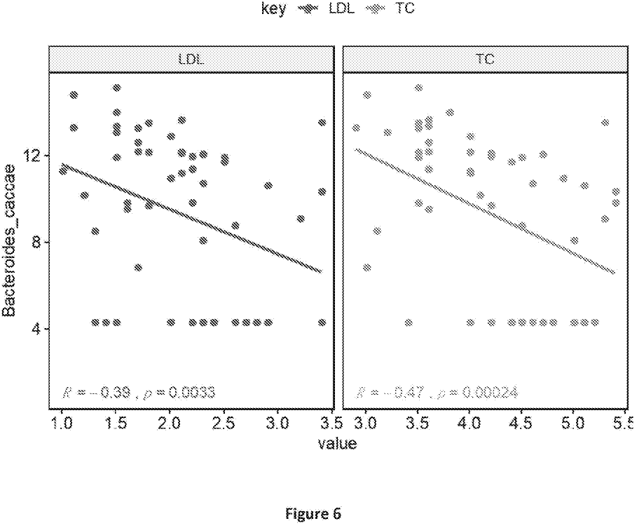

In addition, the species Bacteroides caccae showed both from inverse correlation with total cholesterol, LDL-C and triglyceride, and positive correlation with HDL-C (FIG. 6), indicating that this species is also a salutary microbe protecting against metabolic diseases and cardiovascular disease.

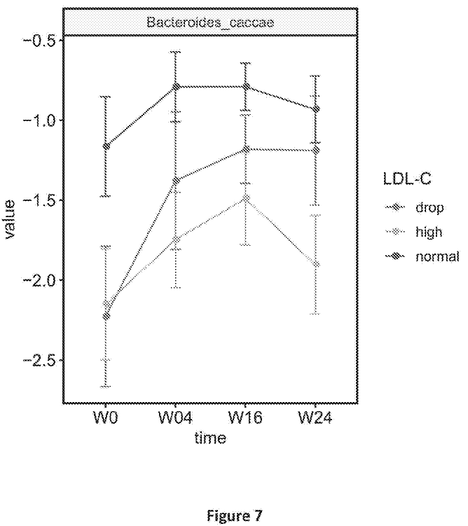

It’s recommended for a patient with type 2 diabetes to keep blood LDL-C level below 1.8 mmol/L. The relative abundance of Bacteroides caccae is significantly higher in patients with normal LDL-C (<=1. 8 mmol/L), compared with patients with high LDL-C (>1.8 mmol/L) (FIG. 7). After intervention, the relative abundance of Bacteroides caccae increased in patients whose LDL-C level returned to normal, while it remained at low level in patients whose LDL-C level remained high (FIG. 7).

In post intervention samples, Streptococcus parasanguinis inversely correlate with TC, Clostridiales bacterium 1_7_47FAA inversely correlate with TC and LDL, Lachnospiraceae bacterium_3_1_57FAA_CT1, Bifidobacterium pseudocatenulatum, Lactobacillus rhamnosus, Ruminococcus obeum, Odoribacter splanchnicus, Blautia hydrogenotrophica, Bacteroides eggerthii, Anaerotruncus colihominis inversely correlate with TG (Tables 4-6).

TABLE 4

Bacterial species for reduction of total cholesterol (TC)

| Bacterial Species | NCBI Taxon ID | Cut-off (relative abundance) |
|---|---|---|
| Lactobacillus mucosae | 97478 | 0.01% |
| Lactobacillus rhamnosus | 47715 | 0.01% |
| Bacteroides caccae | 47678 | 0.012% |
| Streptococcus parasanguinis | 1318 | 0.115% |
| Clostridiales bacterium 1_7_47FAA | 457421 | 0.01% |

TABLE 5

Bacterial species for reduction of low-density lipoprotein cholesterol (LDL-C)

| Bacterial Species | NCBI Taxon ID | Cut-off (relative abundance) |
|---|---|---|
| Megasphaera micronuciformis | 187326 | 0.01% |
| Klebsiella pneumoniae | 573 | 0.008% |
| Bacteroides caccae | 47678 | 0.012% |
| Clostridiales bacterium 1_7_47FAA | 457421 | 0.01% |

TABLE 6

Bacterial species for reduction of triglyceride (TG)

| Bacterial Species | NCBI Taxon ID | Cut-off (relative abundance) |
|---|---|---|
| Bacteroides caccae | 47678 | 0.012% |
| Lachnospiraceae bacterium_3_1_57FAA_CT1 | 8026 | 0.01% |
| Bifidobacterium pseudocatenulatum | 47715 | 0.255% |
| Lactobacillus rhamnosus | 40520 | 0.01% |
| Ruminococcus obeum | 28118 | 2.021 % |
| Odoribacter splanchnicus | 53443 | 0.01% |
| Blautia hydrogenotrophica | 28111 | 0.01% |
| Bacteroides eggerthii | 169435 | 0.01% |
| Anaerotruncus colihominis | 8026 | 0.004% |

All patents, patent applications, and other publications, including GenBank Accession Numbers or equivalents, cited in this application are incorporated by reference in the entirety for all purposes.

### References

1. Bolger AM, Lohse M, Usadel B. Trimmomatic: a flexible trimmer for Illumina sequence data. Bioinformatics 2014;30:2114-2120.

2. Segata N, Izard J, Waldron L, et al. Metagenomic biomarker discovery and explanation. Genome biology 2011;12:R60.

3. Langmead B, Salzberg SL. Fast gapped-read alignment with Bowtie 2. Nature methods 2012;9:357.

4. Grundy SM, Stone NJ, Bailey AL, et al. 2018 AHA/ACC/AACVPR/AAPA/ABC/ACPM/ADA/AGS/APhA/ASPC/NLA/PCNA Guideline on the Management of Blood Cholesterol: Executive Summary: A Report of the American College of Cardiology/American Heart Association Task Force on Clinical Practice Guidelines. Circulation 2019;139:e1046-e1081.

5. Magnusdottir S, Heinken A, Kutt L, et al. Generation of genome-scale metabolic reconstructions for 773 members of the human gut microbiota. Nat Biotechnol 2017;35:81-89.

What is claimed is:

1. A method for reducing bodyweight, reducing low-density lipoprotein (LDL)-cholesterol, reducing total cholesterol, reducing triglyceride, or increasing high-density lipoprotein (HDL)-cholesterol in a subject, comprising introducing into the subject’s gastrointestinal tract an effective amount of one or more bacterial species set forth in Tables 2-6.

2. The method of claim 1, further comprising a step of measuring the level of the one or more bacterial species in a stool sample taken from the subject before the introducing step and a step of measuring the level of the one or more bacterial species in another stool sample taken from the subject after the introducing step.

3. The method of claim 2, wherein the level of the one or more bacterial species after the introducing step is increased compared to the corresponding level of the one or more bacterial species before the introducing step.

4. The method of claim 1, wherein the introducing step comprises FMT or administration to the subject a composition comprising cultured and/or concentrated the one or more bacterial species.

5. The method of claim 1, further comprising a step of determining the percentage relative abundance of the one or more bacterial species in a stool sample taken from the subject after the introducing step.

6. The method of claim 5, wherein the percentage relative abundance of the one or more bacterial species after the introducing step is greater than the corresponding cut-off value in Tables 2-6.

7. The method of claim 1, wherein an effective amount of bacterial species Enterobacter cloacae is administered to the subject, thereby increasing the level of the bacterial species Enterobacter cloacae in the subject’s gastrointestinal tract and increasing high-density lipoprotein (HDL)-cholesterol in the subject.

8. The method of claim 7, further comprising a step of measuring HDL-C level in a blood sample taken from the subject before the administering step and a step of measuring HDL-C level in another blood sample taken from the subject after the administering step.

9. The method of claim 1, wherein an effective amount of one or two bacterial species set forth in Table 2 is administered to the subject, thereby increasing the level of the one or two bacterial species in the subject’s gastrointestinal tract and reducing body weight of the subject.

10. The method of claim 9, further comprising a step of measuring the subject’s bodyweight before the administering step and a step of measuring the subject’s bodyweight again after the administering step.

11. The method of claim 1, wherein an effective amount of one or more bacterial species set forth in Tables 3 and 5 is administered to the subject, thereby increasing the level of the one or more bacterial species in the subject’s gastrointestinal tract and reducing LDL-C level in the subject.

12. The method of claim 11, further comprising a step of measuring LDL-C level in a blood sample taken from the subject before the administering step and a step of measuring LDL-C level in another blood sample taken from the subject after the administering step.

13. The method of claim 1, wherein an effective amount of one or more bacterial species set forth in Table 4 is administered to the subject, thereby increasing the level of the one or more bacterial species in the subject’s gastrointestinal tract and reducing total cholesterol level in the subject.

14. The method of claim 13, further comprising a step of measuring total cholesterol level in a blood sample taken from the subject before the administering step and a step of measuring total cholesterol level in another stool sample taken from the subject after the administering step.

15. The method of claim 1, wherein an effective amount of one or more bacterial species set forth in Table 6 is administered to the subject, thereby increasing the level of the one or more bacterial species in the subject’s gastrointestinal tract and reducing triglyceride level in the subject.

16. The method of claim 15, further comprising a step of measuring triglyceride level in a blood sample taken from the subject before the administering step and a step of measuring triglyceride level in another stool sample taken from the subject after the administering step.

17. The method of any one of claims 1-16, wherein the administering step comprises oral administration or direct delivery to the subject’s gastrointestinal tract.

18. The method of claim 2 or 5, wherein the level of the one or more bacterial species is determined by quantitative polymerase chain reaction (PCR).

19. A kit comprising (1) a first composition comprising donor stool; and (2) a second composition comprising an effective amount of one or more bacterial species set forth in Tables 2-6.

20. The kit of claim 19, wherein the first composition comprises donor stool that has been dried and placed in a capsule for oral ingestion.

21. The kit of claim 19, wherein the first composition comprises donor stool that has been formulated as a solution, suspension, semi-liquid, or paste for delivery by oesophago-gastro-duodenoscopy (OGD), sigmoidoscopy, or enema.

22. The kit of claim 19, further comprising, in a third composition an effective amount of an anti-bacterial agent that reduces total bacterial load in a subject’s gastrointestinal tract.

23. A method for identifying a suitable donor for FMT, comprising the step of determining level of the one or more bacterial species set forth in Tables 2-6 in a stool sample obtained from a candidate.

24. The method of claim 23, wherein the level of the one or more bacterial species set forth in Tables 2-6 is a percentage relative abundance.

25. The method of claim 24, wherein the level of the one or more bacterial species set forth in Tables 2-6 is greater than the corresponding cut-off value in Tables 2-6 and the candidate is identified as a suitable donor for FMT.

26. The method of claim 25, further comprising obtaining stool material from the candidate for use in FMT.

27. The method of claim 25, further comprising determining total bacterial load in the stool sample.

28. The method of claim 23, wherein the level of the one or more bacterial species set forth in Tables 2-6 is determined in a first stool sample obtained from a first candidate and in a second stool sample obtained from a second candidate.

29. The method of claim 28, wherein the first candidate has a higher level of each of the one or more bacterial species set forth in Tables 2-6 than the second candidate and is deemed to be a more suitable FMT donor than the second candidate.

30. A method for improving FMT efficacy, comprising introducing an effective amount of the one or more bacterial species set forth in Tables 2-6 into a composition intended for use in transplantation prior to FMT.

31. The method of claim 30, wherein after the introducing step the level of the one or more bacterial species set forth in Tables 2-6 is greater than the corresponding cut-off value in Tables 2-6.

32. The method of claim 30, further comprising performing FMT using the composition.
